(19) United States
(12) Patent Application Publication
(10) Pub. No.: US 2017/0252135 A2
(43) Pub. Date: Sep. 7, 2017

(54) GINGIVAL INDEXING DEVICE AND METHOD FOR INDEXING THE GINGIVA

(71) Applicant: Heraeus Kulzer GmbH, Hanau (DE)

(72) Inventors: David J. REAY, Nocton Lincoln (GB); Harald KUBIAK-ESSSMANN, Nidderau (DE)

(73) Assignee: Heraeus Kulzer GmbH, Hanau (DE)

(21) Appl. No.: 15/115,378

(22) PCT Filed: Jan. 28, 2015
(86) PCT No.: PCT/EP2015/051693

§ 371 Date: Jul. 29, 2016

(51) Int. Cl. A61C 13/01; G01J 3/50; A61C 13/08; A61C 19/10; A61B 5/103; A61B 5/00

(52) U.S. Cl. CPC A61C 13/01; A61B 5/1034; A61B 5/4552; A61C 13/082; A61C 19/10; G01J 3/508

(57) ABSTRACT

Gingiva indexing devices and methods with various defined red colour regions have a colour grey card or grey card assigned to it for detecting the gingiva status, in particular for producing a gingival restoration. The devices and method comprise an analysis of at least one region of the gingiva status with regard to its shades of red, the position of the shades of red, the colour profile of the shades of red, their geometry and/or their morphology. The analysis can take place manually and/or digitally, and gingiva information is obtained, in particular in the form of a gingiva map, and optional production of a virtual or real model of a gingival restoration as a reproduction of the missing part of the gums and/or part of gingiva.

This application is a 371 application of PCT/EP2015/051693, filed Jan. 28, 2015, which claims foreign priority benefit under 35 U.S.C. 119 of German Application No. DE 10 2014 101 059.0 filed Jan. 29, 2014.

The invention relates to a gingiva indexing device with various defined red colour regions (shades of red) and a method for detecting the gingiva status, in particular for producing a gingival restoration, comprising an analysis of at least one region of the gingiva status with regard to its shades of red, the position of the shades of red, the colour profile of the shades of red, their geometry and/or their morphology, whereby the analysis takes place manually and/or digitally, and gingiva information is obtained, in particular in the form of a gingiva map. Optionally, a virtual or real model of a gingival restoration as a reproduction of the missing parts of the gums or part of gingiva is produced. A gingival restoration is also called a gingival mask.

There are various types of gingival restorations or gingival masks. There are gingival masks for use in the mouth of the patient for replacement of age- or disease-related receding gingiva as well as gingival masks as an aid for the dental technician for simulating preparation limits on the plaster model. Also, there are gingival restorations or gingival masks as part of a dental prosthesis. The dental prosthesis can comprise fixed or removable dentures. In this context, the removable denture can be affixed, for example, on implants incorporated into the jaw and on holding elements, such as snap fasteners or bars, attached to them. One disadvantage of said dental prostheses with dentures is that the gingival restoration on the denture aiming to conceal the recession of gingiva and, optionally, loss of jaw bone (atrophy) is single-coloured.

Type 1: Gingival masks can be used if there is disease- or age-related gingival recession. In this case, the progressing visibility of the dental necks makes the teeth look unnaturally long which can lead to serious cosmetic problems. In the presence of disease-related gingival recession, the focus is on treating the underlying disease. Once the gingiva is stable and free of inflammation again, the gingival recession can be concealed with a so-called gingival mask. A gingival mask is an artificial gingiva, which usually consists of a flexible polymer. The gingival mask is a removable gingiva imitation, which usually can be inserted and removed by the patient. Likewise, gingival recession and/or a gingival deficit in the dentition involving tooth implants can be concealed by means of a gingival mask and/or gingival restoration. In this case, the gingival restoration can be part of a prosthetic management with a denture.

Until now, said gingival restoration and/or mask is fabricated individually on a plaster model by the dental technician and then fitted inside the patient's mouth by the dentist. In the presence of large gingival defects, e.g. due to advanced periodontitis, a removable gingival mask is often the only available solution for reproducing or restoring the original aesthetic. After taking an impression, plastic material is used in the dental laboratory to fabricate a wafer-thin gingiva-coloured mask that touches precisely against the teeth. Wearing said gingival mask is usually very comfortable for the patient. Moreover, the aesthetic appearance and phonetics (pronunciation) are improved significantly. For cleaning of the teeth, the mask is easy to remove and to insert afterwards.

Type 2: In the field of dental engineering, a permanently soft material imitating the gingiva on the plaster model is also called gingival mask. An aid of this kind can be used to reflect the optical, spatial, and aesthetic relationships in the mouth very well. However, it is a disadvantage of the gingival masks or gingival restorations of the prior art that they are monochrome in colour. Thus far, gingival restorations are fabricated from a single shade of red such that there is no economical method for transferring the naturally occurring shades of red of the gingiva.

DE 8121341 U1 discloses a gingival mask for producing and fitting the denture to the gingiva region and thus discloses an aid for the dental technician for simulating the preparation margin in the model. The gingival mask is produced by filling flowable, curable material into the intervening spaces of the plaster model. A single-coloured gingival mask is obtained.

DE 2744662 C3 discloses a gingival mask structured in the way of natural gingiva and a method for the production thereof from a silicone rubber. For production reasons, a dough mass of a silicone material is placed in a pressing mould such that a single-coloured gingival mask is obtained.

The production-related monochromaticity is an essential aesthetic disadvantage of the gingival restorations of the prior art. Moreover, thus far there is no method for detecting and analysing the gingiva status with regard to different shades of red and their colour profiles, since only an impression of the dentition status is taken thus far.

An assignment of colours in the field of dentistry is only known for colour shades of teeth thus far.

It was the object of the invention to provide an economical method for detecting the shades of red of the gingiva status, colour profiles of the shades of red and, in particular, in combination with the morphology and geometry of the gingiva status. Moreover, a device and a software solving the object for implementing the method were to be found. It was also an object to provide a very aesthetic- and natural-looking gingiva for a partial or total prosthesis.

The objects are solved according to the subject matters of claims 1, 8, 11, and 13 and 14, whereby advantageous embodiments are illustrated in the sub-claims and, in detail, in the description.

One subject matter of the invention is a gingiva indexing device comprising a) at least one colour region, whereby the colour region comprises at least one shade of red, and b) the indexing device has at least one colour grey card and/or one grey card assigned to it, in particular the indexing device comprises at least one region with a colour grey card and/or a grey card.

In this context, the colour grey card or grey card can, for example, correspond to the base or the two dimensional element, in particular planar element, of the indexing device. The colour grey card and/or grey card can just as well be arranged in an area that is analogous to a colour region. A kit comprising a gingiva indexing device that comprises at least one colour region, whereby the colour region comprises at least one shade of red colour or shade of red, respectively, whereby the kit comprises a colour grey card and/or a grey card, is also a subject matter of the invention.

Preferably, a colour region comprises a shade of red and is, in particular, separated from other colour regions of the indexing device. Alternatively, a colour region can just as well comprise different defined shades of red in a defined arrangement or in a typical colour profile. To attain an excellent result in terms of the detection of the gingiva status by means of the indexing device, the colour regions can preferably be three-dimensional colour bodies, which preferably reflect typical red colour profiles and/or a typical morphology of a region of a gingiva.

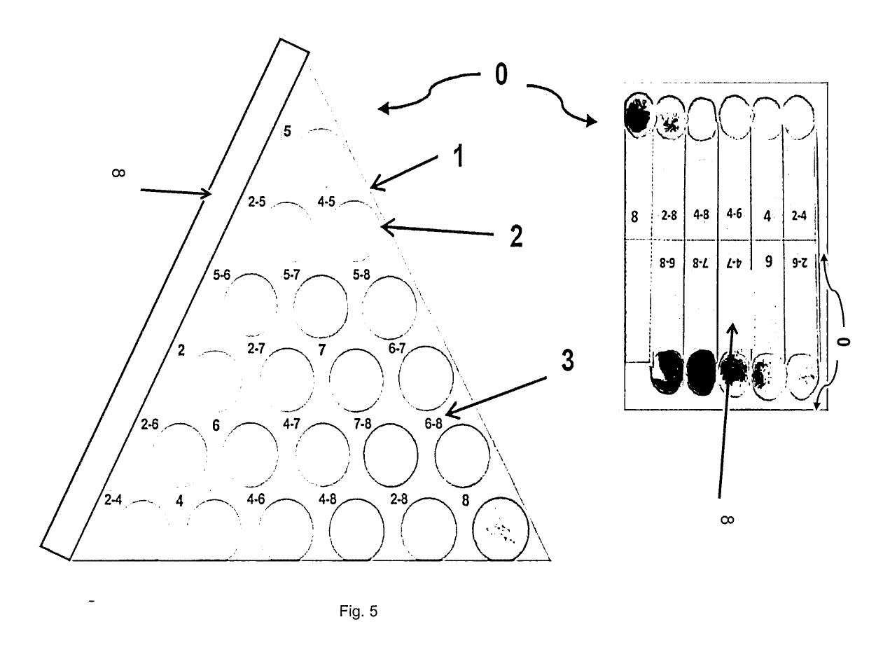

An indexing device, whose colour regions each are assigned to a colour mixing instruction, is also a subject matter of the invention. The colour mixing instruction is evident, for example, from the indexing device in FIG. 5, whereby 5-6 means that the colour is available by mixing dental colours 5 and 6 at a weight or volume ratio of 1:1.

The colour grey card and/or grey card according to the invention serves an image processing software as a reference for determining the colour temperature of the light, the white balance and/or for assessment of a colour distortion adaptation of the light. According to the invention, it is therefore not necessary for the colour grey card or grey card to correspond to a card, but it is important that the colour grey card or grey card bears the information required for calibration or performance of a white balance and/or of a colour distortion assessment or of a colour distortion adaptation.

Particularly preferably, each colour region of the indexing device has at least one coding assigned to it, preferably each independently. The coding is used to provide a colour mixing instruction for colour reproduction of the gingiva. The coding or coded colour regions comprise coding, such as letter codes, letter and number codes, number codes, character codes, such as, preferably, bar codes, line codes, 2D or 3D bar codes as well as further codings known to a person skilled in the art. The coding can also be called colour indicator. The colour mixing instruction is used for true-to-nature generation of the colours of the gingival restoration. A gingival restoration according to the invention therefore comprises a true-to-nature reproduction of the colours, colour profiles of shades of red of the natural gingiva, preferably at least 2 to 5,000 different shades of red, particularly preferably 5 to 2,000 different shades of red.

A gingiva indexing device is a device allowing gingival regions, in particular human gingival regions, to be mapped by means of defined red colour regions, i.e. to spatially and geometrically detect, analyse, and document these regions. The gingiva indexing device allows, in particular, for reproducible detection, analysis and/or documentation of the gingiva status in terms of the shades of red, their colour profiles and/or the morphology of the gingiva status. The gingiva indexing device enables the topographical assignment of the colours in the restoration. The indexing device and the method according to the invention allow for detection of the gingiva colours, i.e. of the gingival characteristics.

A gingiva indexing device can be intended for multiple or single use. For example, an indexing facility can be a tear-off booklet with different strips with colour regions. Likewise, a kit with colour bodies is conceivable, whereby the colour bodies are held to the gingiva by means of an instrument and/or forceps. In general, a gingiva indexing device can take any reasonable design.

A colour region corresponds to a delimited two- or three-dimensional region comprising at least one shade of red.

A gingival restoration is a reproduction of the missing part of gingiva, missing gingiva and/or missing gingival part, in particular for a region of gingiva. Likewise, the gingival restoration can advantageously be a part of a prosthetic management with a denture.

Graded colour bodies are colour bodies, in particular three-dimensional colour bodies, with at least one coded colour region. The gingiva indexing device comprising graded colour bodies can, for example, be a box with separate compartments, in which a multitude of each type of the graded colour bodies is provided in the corresponding compartments. Advantageously, the graded colour bodies are designed for single use.

An indexing device comprising 1 to 5,000 colour regions is also a subject matter of the invention, whereby each colour region, independent of each other, comprises a different defined shade of red. In particular 2 to 2,000 colour regions, advantageously comprising 2 to 100 colour regions, preferably with 5 to 100 colour regions, particularly preferably with 10 to 100 colour regions, more preferably with 6-15 or, just as well, 21 colour regions, in particular with at least one shade, preferably each colour region comprising one shade of red that deviates from the shades of red of the other colour regions. Particularly preferably, a colour region comprises a typical colour profile of shades of red of a region of gingiva.

According to a preferred alternative, the indexing device comprises at least one colour region, in particular at least 2 to 2,000 colour regions, which each are coloured bodies. Advantageously, each colour region independently is a coloured body, preferably the coloured body can be detached from the indexing device. A coloured body can comprise polymers, ceramic materials, composite materials, glasses or hybrid materials.

Moreover, the colour regions are preferably positioned in a defined arrangement with respect to each other on the indexing device. In the scope of the invention, an indexing device comprises colour regions that are connected on, in, at, affixed or detachably, removably, etc. with the device or are attached to it. It is preferred in this context to have the colour regions of the indexing device be arranged ranging from a bright shade of red to a dark shade of red and/or from a red with yellow portions to a red with blue portions. Advantageous colour profiles comprise a Kodak colour scale, RGB model, CMYK model, Pantone, HKS colour scheme, RAL colour system or other colour systems comprising shades of red or nuances of red familiar to a person skilled in the art.

Advantageous indexing devices comprise colour regions that each are coloured bodies and advantageously are arranged on an element. Suitable, as elements, are all two- or three-dimensional elements, such as planar elements, elongated three-dimensional bodies, rods, tubes, films, instruments, three-dimensional elements. In this context, the two-dimensional elements can advantageously be made of flexible material or rigid material. Advantageously, the colour regions, in particular the coloured bodies, are attached to the element such as to be detachable or removable. The element as such, preferably can be subdivided, for example by cutting, by tearing, by pulling apart, etc. The colour regions or elements can just as well adhere to the gingiva to simplify the analysis. Accordingly, the invention also comprises bondable colour regions or elements.

Advantageous planar elements can be present as triangular, rectangular, strip-shaped; square, circular, oval elements. Likewise, an element can be present as polygon, such as a pentagon, hexagon, heptagon, octagon, etc., and comprises at least one colour region that is arranged on the element, whereby different colour regions are arranged on different elements or on one element.

The coloured elements, in particular bodies, can just as well be of any three-dimensional shape such as, for example, but not conclusively, polyhedra, oval, rod-shaped, sections of a spherical surface.

According to a particularly preferred embodiment,

a) the colour regions, in particular the coloured bodies, are arranged on an essentially triangular planar element, whereby the colour profile of the shade of red changes along a leg of the triangle from a red with yellow portions (red-yellow) to a red with blue portions (red-blue) and/or changes from a bright shade of red to a dark shade of red, in particular the red-blue shade and the dark shade of red are displayed as shades in a colour region, or

b) the indexing device is a planar element, in particular a square or rectangular planar element, comprising detachable colour bodies, removable colour bodies or detachable holders each with at least one colour body, detachable holders with at least one colour region per holder, removable regions with at least one colour region or removable regions with at least one colour body, whereby the holders or regions are advantageously formed by a perforation of the planar element, in particular removable strips each with at least one colour body or removable strips each with at least one colour region, whereby the planar element preferably is dividable, or

c) the indexing device is part of a kit comprising colour bodies, in particular graded colour bodies, in particular colour bodies whose shade of red comprises a nuance from yellow toward blue and from bright red toward dark red, whereby the colour bodies can be attached to or on an element, in particular at least one coding is assigned to each of the colour bodies. Advantageously, the coding can be attached to the colour bodies. The colour bodies according to c) can, for example, be attached to a dental instrument, for example an instrument can be pressed into the colour body and the colour body can be impaled or held to the gingiva with forceps. The term, removable, shall be understood to mean separate, tear off, etc. Alternatively, the colour regions or colour bodies can be re-gluable.

According to an alternative, the indexing device is an indexing device for single use. Advantageously, the device is biologically degradable.

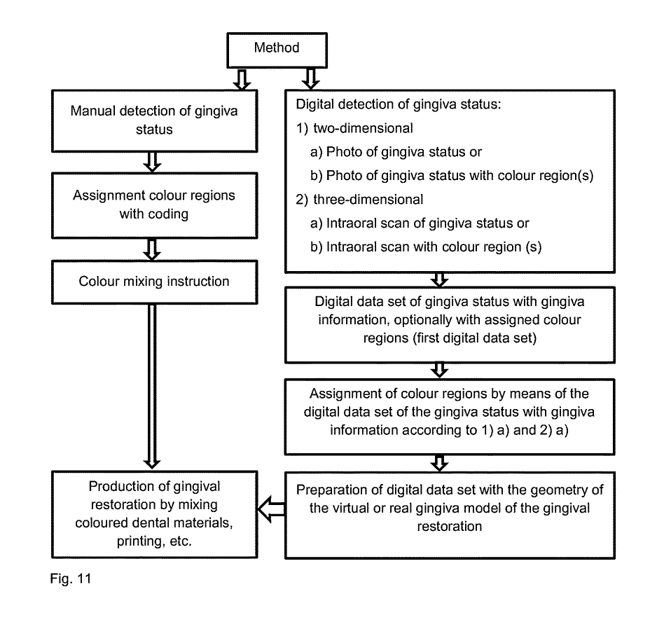

A kit comprising a gingiva indexing device and a write-on gingiva template is also a subject matter of the invention. A write-on gingiva template shall be understood to be a template according to FIGS. 9a) and b) and FIGS. 10a) and b). FIGS. 10a) and b) depict labelled gingiva templates. Alternatively, the write-on gingiva template can just as well be a three-dimensional model of the gingiva status or of the dentition or a part of such models. A write-on gingiva template shall just as well be understood to be a virtual motif of the gingiva that is displayed in a software and can have a coding added to it. The gingiva template can just as well be a two- or three-dimensional virtual model.

The object of the kit can just as well be a permanently soft dental material, a composite material or a ceramic dental material for producing the gingival restoration.

Another subject matter of the invention is a method for detecting the gingiva status and, in particular, for producing a gingival restoration, in which
A) an analysis of at least one region of the gingiva status, in particular of the remaining gingiva, takes place with respect to its shades of red, the position of the shades of red and/or the colour profile of the shades of red and, optionally, the geometry, in particular taking into consideration the tooth status, and/or morphology, whereby the analysis takes place in digital manner; and
B) gingiva information is obtained, in particular gingiva information is obtained in the form of a gingiva map, whereby the analysis of the gingiva status advantageously takes place with regard to at least 2, 3, 4, 5, 6, 7 up to 2,000 shades, in particular digital gingival information being obtained, preferably the gingival information being obtained as a digital data set, preferably as a first digital data set; and
C) optionally, a virtual or real model of a gingival restoration, i.e. of a reproduction of the missing part of gingiva, is produced and, in particular, a gingival restoration, in particular of the missing part of gingiva, is produced.

Method for detecting the gingiva status, in particular for preparing a virtual or real gingival restoration, in which
A) an analysis of at least one region of the gingiva status takes place with respect to its shades of red, the position of the shades of red and/or the colour profile of the shades of red and, optionally, the geometry and/or morphology;

whereby analysis A) takes place in digital manner; and
a.1) whereby at least one region of the gingiva status is detected together with a colour grey or grey card and, optionally, together with at least one shade of red of an indexing device, with the detection taking place, in particular by means of photographing or scanning together with a colour grey or grey card;
a.2) whereby digital gingiva information is obtained as a digital data set, in particular as a first digital data set, and, optionally, the data set is provided, stored and/or transmitted;
a.3) a white balance and/or a colour distortion adjustment is performed on the digital data set, in particular using a software, such as an image processing software, and a colour-adapted digital data set (calibrated data set) is obtained;
a.4) the data set is analysed for colour shades and/or colour profiles; and
B) gingiva information is obtained, and, optionally,
b.1) the gingiva information is assigned to ready-to-use, in particular coded, dental colours and/or a colour mixing instruction for dental colours, and/or
b.2) the digital data set with the geometry of the virtual or real gingiva model of the gingival restoration is prepared, optionally through the use of the colour-adapted digital data set with gingiva information, and optionally, the tooth status and/or the region of the part of the face, and, optionally,
C) a virtual or real model of a gingival restoration, i.e. a reproduction of the missing part of gingiva, is produced.

Production of a gingival restoration, whereby the order of steps can deviate from a.3, a.4, b.1 and b.2, for example the order of steps being b.2, a.3, a.4, b.1 or any other reasonable order. It can be reasonable to perform step b.2, in which the data set comprising the geometry of the virtual or real gingiva model is provided, ahead of steps a.1, a.2, a.4, and/or a.3, such that the colour-adapted data set b.2 is added to the geometrical data b.2.

Advantageously, in this context, the detected shades of red, the positions of the shades of red as well as the positions of the shades of red with respect to each other, the geometry of the gingiva, as well as the tooth status and/or the morphology of the gingiva, in particular of the remaining gingiva, in at least two different regions of the gingiva status are used and/or transferred by means of the gingiva information obtained in a) for producing the gingival restoration.

For example, the gingiva information is transferred to an impression of the dentition status, a plaster model of the dentition status or a write-on gingiva template.

Preferably at least 2 to 5,000 shades of red, in particular 5 to 30 shades of red (colour shades), particularly preferably at least 10 to 25 shades of red are detected as well as, advantageously, those of the interdental papilla (papilla interdentalis).

Advantageously, the gingiva information can be detected at a resolution of 50 dpi, preferably 150 dpi, in particular higher than/equal to 300 dpi, preferably at a good resolution of higher than/equal to 600 dpi (pixels per inch) up to a high resolution of higher than/equal to 1200 dpi or higher. Advantageously, the gingiva information can, in addition or alternatively, be detected and, preferably, reproduced, at a level of detail of less than/equal to 100 μm, in particular less than/equal to 50 μm, particularly preferably down to less than/equal to 30 μm, down to less than/equal to 20 μm.

The triangle-shaped gingiva between the teeth is called “interdental papilla” (papilla interdentalis) and the borderline between the gingiva and the shiftable dark red oral mucosa is called mucogingival line or a mucogingival border. It is advantageous to analyse these regions at particular detail and to capture the corresponding gingival information.

Another subject matter of the invention is a method, in which analysis A) takes place in digital manner,
in that the at least one region of the gingiva status, in particular of the remaining gingiva, and, optionally, a region of the face is detected digitally, two- or three-dimensionally, in particular with the region being scanned or photographed, preferably together with a colour grey card and/or grey card for calibration, whereby digital gingiva information is obtained as a digital data set, in particular as a first digital data set, preferably with colour information being obtained and, optionally, in addition three-dimensional information being obtained, or
in that a two- or three-dimensional detection of at least one region of the gingiva status and of the tooth status and, optionally, of a region of the face and at least one part of a colour region with shade of red of the indexing device takes place, whereby the detection takes place in conjunction with the detection of at least one part of a colour grey card and/or grey card for calibration, and digital gingiva information is obtained as a digital data set, in particular as a first digital data set.

Alternatively, at least one detection of a region of the gingiva status and of the tooth status and of a red colour region and, optionally, of a region of the face in conjunction with at least one part of a colour grey card and/or grey card takes place, in particular takes place by scanning or photographing, whereby digital gingiva information is obtained as a digital data set.

Preferably, a two- or three-dimensional detection by means of an intraoral scan or by means of photographing takes place. According to this alternative, what is comprised is a method, in which the colour regions of the indexing device are held to the gingiva and the assignment is detected by means of a photograph or intraoral scan. The colour or grey card is detected in the same process for later calibration of the shades in the data set.

The digital data thus obtained can then be transmitted easily by the dentist to the dental technician for production of the gingival restoration. It is a particular advantage of the method according to the invention that it is feasible to prepare a virtual model of the gingival restoration and transmit it to the dentist and the patients before producing a real gingiva model or the gingival restoration. This allows to come to an agreement with the dentist and the patient regarding the aesthetic features at an early time.

The analysis of the detected gingiva information, in particular in the form of a digital data set, takes place by means of a manual or automatic analysis and assignment to colour regions of an indexing device or of a write-on gingiva template.

Another subject matter of the invention is a method, in which analysis A) takes place in digital manner, 1) in that at least one region of the gingiva status and, optionally, one region of the face is detected while using a colour grey card and/or a grey card for calibration, whereby the region of the gingiva is digitally detected, two- or three-dimensionally, in conjunction with the card, in that the region and at least one part of the card are scanned or photographed, whereby digital gingiva information is obtained as a digital data set, or 2) in that a two- or three-dimensional detection of at least one region of the gingiva and, optionally, of the tooth status and, optionally, of a region of the face and at least one colour region with a shade of red of an indexing device takes place, whereby the detection takes place in conjunction with the detection of at least one part of a colour grey card and/or grey card for calibration, with the detection taking place, in particular, by scanning or photographing, whereby digital gingiva information is obtained as a digital data set.

Alternatively, an impression compound or a model of the gingiva can be digitally detected in three-dimensional manner, and a digital data set of geometric data is obtained. Finally, said data set can be provided with the digital gingiva information.

The digital data can be stored, transferred as well as modified in order to produce a model of a virtual gingival restoration.

Alternatively or in addition, analysis (ii) can take place manually, in that 3) a manual two- or three-dimensional detection of at least one region of the gingiva takes place or at least a detection of a region of the gingiva and of the tooth status or at least a detection of a region of the gingiva and of a colour region of an indexing device or at least a detection of a region of the gingiva and of the tooth status and of one colour region takes place, and manual gingiva information is obtained. According to this variant, gingiva information determined by means of the indexing device can be recorded as written notes, for example on the gingiva template.

Another subject matter of the invention is a method, in which the gingiva is analysed: a) manually through the use of an indexing device, in that the gingiva is examined with the indexing device for colour shades and/or colour profiles, in particular in the region of the gingiva at the front teeth region (anterior) and/or lateral teeth region (posterior) for shades and/or colour profiles, in particular mandibularly and/or maxillary, with the gingiva particularly preferably being analysed for (i) colour profiles of the papillae, crestal, in the region of the sulcus, with, additionally or alternatively, the (ii) geometry of the gingiva being analysed, in particular for the position of the papillae, the crestal status, the tooth width, the alveolar ridge and/or the tooth status, such as the visibility of the dental necks and/or the (iii) morphology of the gingiva being analysed, and gingiva information being obtained, in particular as manual gingiva information.

In a preferred method, analysis (i) takes place digitally through the use of a software, in that a white balance and/or balance of the colour distortion, for example colour distortion assessment, in correlation to the colour grey card or grey card is performed through the use of a software on the digital data set of the digital gingiva information, and a colour-adapted digital data set is obtained.

Said data set is a calibrated data set. The colour-adapted digital data set of the gingiva and, optionally, of the tooth status is analysed manually with a cursor or automatically for shades and/or colour profiles, particularly preferably the gingiva is analysed for (i) colour profiles of the papillae, crestal, in the region of the sulcus, with, additionally or alternatively, (ii) the geometry of the gingiva being analysed, in particular for the position of the papillae, the crestal status, the tooth width, the alveolar ridge and/or the tooth status, such as the visibility of the dental necks and/or the (iii) morphology of the gingiva being analysed. In the process, gingiva information is obtained and, optionally, the gingiva information and/or the gingiva regions each independently have one or more defined, ready-made, dental colours, in particular coded colours, and/or one or more colour mixing instructions of dental colours assigned to them, in particular from which a dental colour or a colour mixing instruction of dental colours can be selected manually or automatically.

According to the invention, it is preferred to analyse the gingiva digitally and, optionally, manually: a) digitally and, optionally, manually through the use of a software, in that the gingiva status and, optionally, the tooth status is examined with the software for colour shades and/or colour profiles, in particular in the region of the gingiva at the front teeth region (anterior) and/or lateral teeth region (posterior) for colour shades and/or colour profiles, in particular mandibularly and/or maxillary, with the gingiva particularly preferably being analysed for (i) colour profiles of the papillae, crestal, in the region of the sulcus, with, additionally or alternatively, (ii) the geometry of the gingiva being analysed, in particular for the position of the papillae, the crestal status, the tooth width, the alveolar ridge and/or the tooth status, such as the visibility of the dental necks and/or the (iii) the morphology of the gingiva being analysed, and gingiva information being obtained, in particular as manual gingiva information. Preferably, the gingiva information is transferred to a virtual gingival template, preferably automatically through the use of a software.

Particularly preferably, the gingiva information is obtained in the form of a gingiva map, in particular as a digital data set. Advantageously, a virtual and/or real model of a gingival restoration can be prepared from said gingival information while taking into consideration the compensation of the gingival recession and/or loss of jaw bone. A gingiva map, in particular with a motif of the gingiva, with assigned, defined, ready-made, coded dental colours or colour mixing instructions for dental colours is particularly preferred. Dental colours shall be understood to be compositions of dental colours for producing a gingival restoration.

According to a particularly preferred alternative, the digital gingiva information of the digital data set a) is assigned to digitally coded colour regions, or b) is assigned to manually coded colour regions of the indexing device, whereby a colour mixing instruction each is being assigned independent of the coding and/or coded colour regions. The colour mixing instruction renders a natural colour reproduction of the gingiva in a gingival restoration feasible.

Another subject matter of the invention is a method for producing a virtual or real gingival restoration, in that (i), in a step prior to the preparation of the virtual or real gingival model, a first digital data set comprising digital gingiva information of a first virtual model of the gingiva status and, optionally, of the tooth status and/or a region of the face is prepared, in particular in conjunction with the detection of a colour grey card and/or grey card for calibration and, optionally, at least one colour region with a shade of red of an indexing device, preferably by means of an intraoral scan or at least one individual photograph taken, for example, with a smart phone. Preferably, the first digital data set corresponds to the data set with digital gingiva information.

Alternatively, a 3D scan of a silicone impression or a 3D scan of a real model, in particular a plaster model, can take place and can be supplemented with digital gingiva information (colour information of the gingiva).

Optionally, (i.0) providing, storing, transmitting of the respective data set is. Moreover, the method can comprise the following steps

(i.1) performing a white balance and/or a balance of the colour distortion and obtaining a colour-adapted digital data set, and, optionally, (i.2) analysing the colour-adapted data set for colour shades and/or colour profiles, and obtaining gingiva information and, optionally, (i.3) assigning the gingiva information to ready-made, in particular coded, dental colours and/or a mixing instruction of dental colours, and, optionally, (ii) preparing, in a next step, the digital data set with the geometry of the virtual or real gingiva model of the gingival restoration and, optionally, through the use of the colour-adapted digital data set or containing the information from the colour-adapted data set, and

(iii) optionally, producing a gingival restoration, with the gingival restoration preferably being produced while compensating for the gingival recession, loss of jaw bone and the above-mentioned red colour profiles as well as, optionally, the morphology. In this context, the order of (i.1 to i.3) and (ii) can be reversed by first performing (ii) and then (i.1 to i.3).

The production preferably involves the use of defined ready-made coded colours and/or the use of the colour mixing instruction or the use of the gingiva map, in particular with defined ready-made coded colours or colour mixing instructions.

Another subject matter of the invention is a method for producing a gingival restoration as well as a gingival restoration obtainable according to said method, in that a detection of the gingiva status takes place according to the method according to the invention and the production of a virtual or real model of a gingival restoration takes place, in that the shades of red, the position of the shades of red, the geometry of the gingiva and/or the morphology of the gingiva in at least two different regions of the gingiva are used and/or transferred, by means of the gingiva information obtained, for production of the gingival restoration.

The gingiva information of the detected gingiva status can be used for production of the gingival restoration and a virtual or real model of a gingival restoration can be simulated and/or prepared taking into consideration and compensating for the gingiva recession and/or the loss of jaw bone as well as aesthetic considerations. Agreement on the virtual model can preferably be reached with the patient before producing the model. Advantageously, the virtual model can be copied into the gingiva status and can be illustrated to the patient.

Another subject matter of the invention is a gingival restoration obtainable according to the method according to the invention. Advantageous gingival restorations are made from a permanently soft dental material, a composite material or a ceramic dental material.

Gingival restorations can be produced, for example, according to a method according to the disclosure of DE 10 2012 012 346 A1 in that the gingiva shades are adjusted during the printing process in accordance with the colour mixing instruction.

Another subject matter of the invention is a digital data set, obtainable according to the method according to the invention, that is stored, in particular, on a data medium.

Another subject matter of the invention is a software for the processing of digital data sets, obtained according to the method according to the invention, in particular on a computer-readable medium, such as a data carrier or a computer, in particular for processing digital data sets comprising gingiva information, preferably for analysis of at least one region of gingiva, in particular the gingiva status, for its shades of red, the position of the shades of red, the colour profile of the shades of red, geometry and/or morphology and for providing gingiva information and, optionally, for assigning coded colour regions, whereby the coding is assigned to a colour mixing instruction and/or for preparing digital data sets of the gingival restoration.

Advantageously, the software allows for analysis of digitally supplied data of the at least one region of gingiva, in particular of the remaining gingiva and/or gingiva status, for its shades of red, the position of the shades of red, the colour profile of the shades of red, colour nuances, geometry, tooth status and/or the morphology, and concurrently provides gingiva information as a digital data set. Optionally, the software allows for assignment of the different regions of the gingiva status to coded colour regions, whereby a colour mixing instruction is assigned to the coding. Alternatively or additionally, the software allows for calculation of digital data sets of a virtual gingival restoration or proposes virtual models of a gingival restoration.

The software can be provided in the form of an application (app, mobile app) on a computer, in particular on a mobile computer, in particular on a smart phone, a digital camera or any other suitable electronic device.

For natural reproduction and production of gingival restorations, these can be produced through the use of the digital data set by means of milling from a body and subsequent printing-on of the coloured gingiva, by means of rapid prototyping, in particular by means of light-curing generative procedures, layer-by-layer from a curable composition of monomers and/or pre-polymers, preferably a radiation-curable composition. Stereolithography, DLP procedures or 3D printing procedures can be used just as well. Alternatively, the classical manual production of a gingival restoration taking into consideration the gingiva information for reproduction of the red aesthetics features is feasible.

The invention is illustrated in more detail based on the following figures without limiting the invention to the examples given.

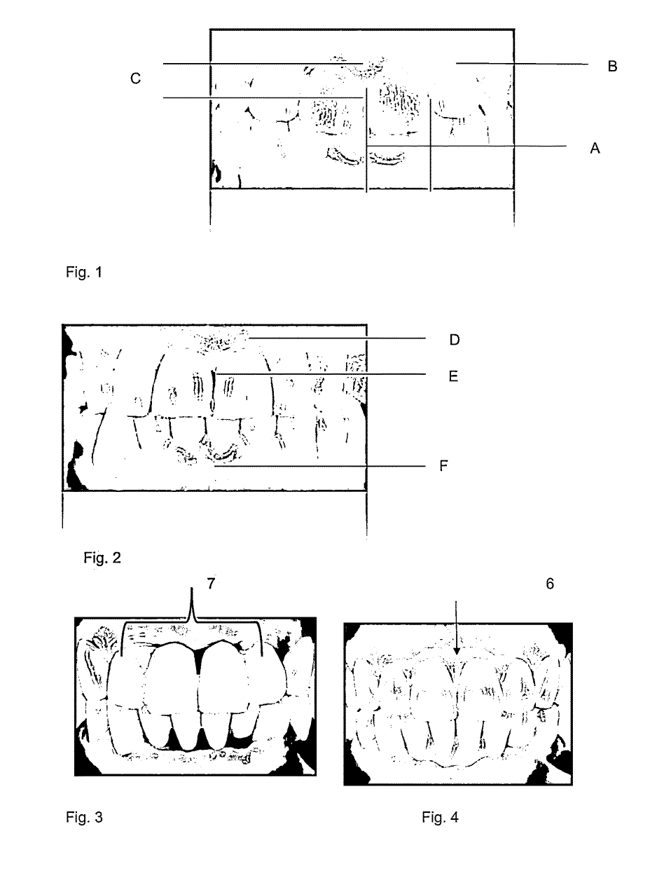

FIGS. 1 and 2 show a part of the regions to be analysed for determining the gingiva status.

FIG. 3 shows a gingiva status with gingival deficit 7, whereas

FIG. 4 shows the mouth with gingival restoration.

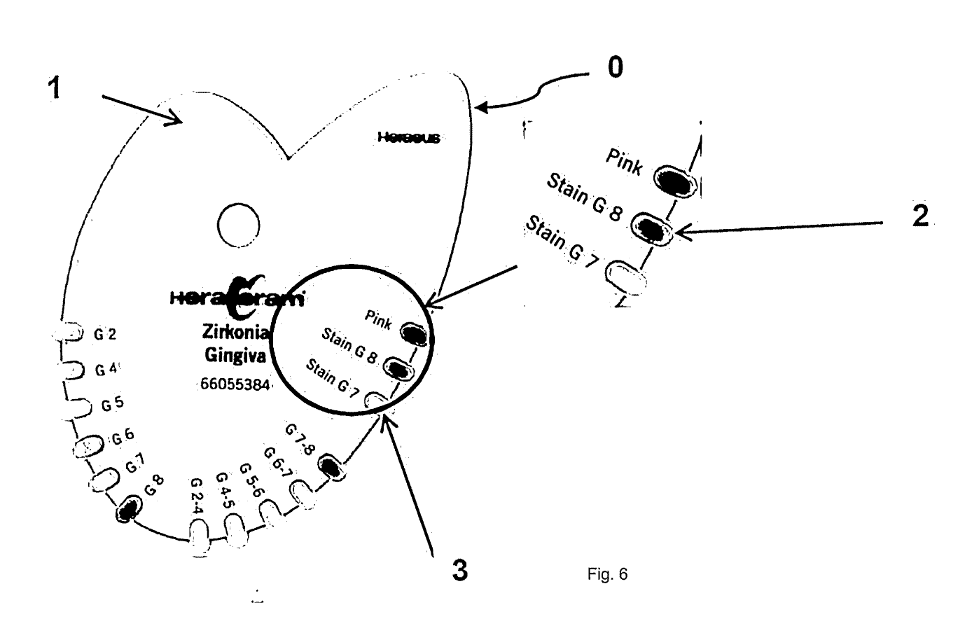

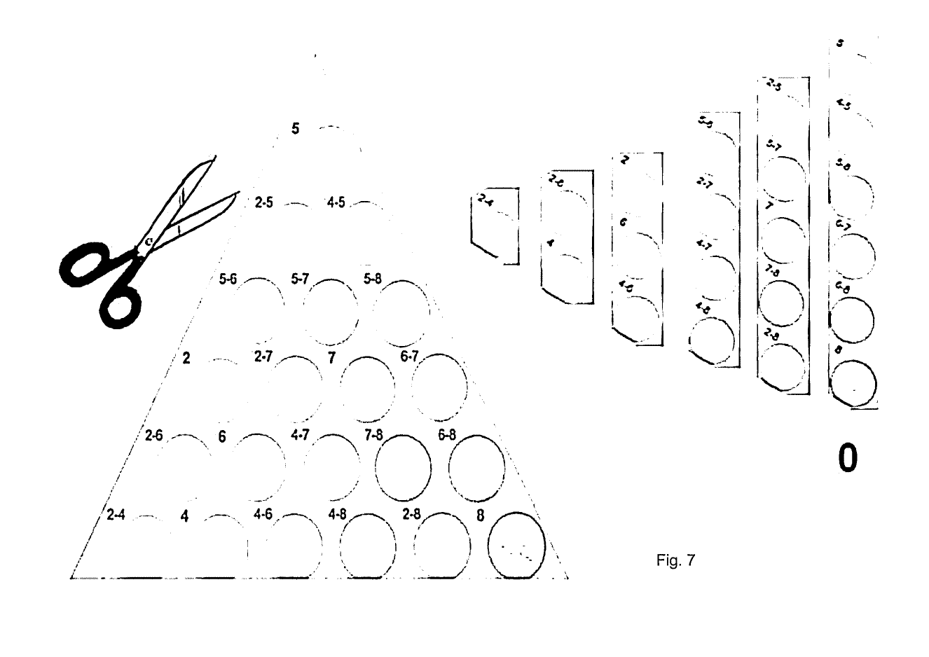

FIGS. 5, 6, and 7 show a gingiva indexing device 0 in the form of an element, in particular a planar element 1 having a multitude of colour regions 2, which are provided as coloured bodies with a defined shade of red in these alternatives. The colour regions 2 are coded 3, whereby the coding has a colour mixing instruction (manual or digital) assigned to it such that the natural reproduction of the different shades of red of the gingiva, their colour profiles, morphology, and geometry are feasible based on the detected colour region and its coding. The reproduction can take place by means of printing, mixing, etc., of plastic materials, plastic materials with a filler content, elastomeric plastic materials, ceramics or composites. FIG. 7 shows an indexing device and one option of separating the colour regions by cutting the device. The indexing device shown is a single-use indexing device.

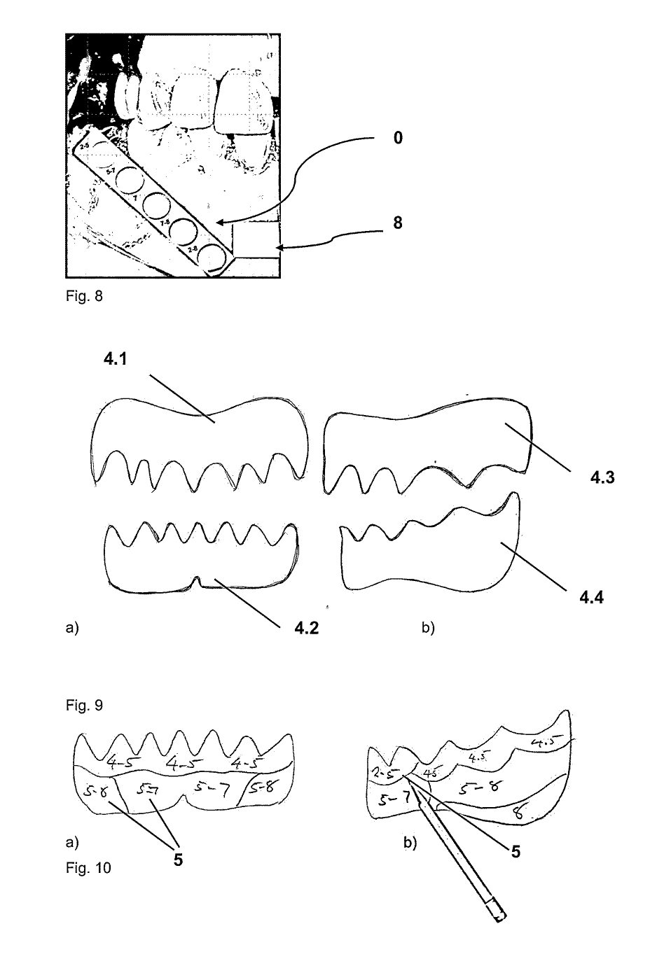

FIG. 8 shows a possible scenario for detection of the shades of red and their colour profiles in that the gingiva is analysed by means of the indexing device and individual colour regions are assigned to defined positions of the gingiva. The analysed and assigned colour regions can then be transferred, in terms of their position, to the write-on gingiva template of FIGS. 9 and 10.

FIG. 10 shows a gingiva map of the shades of red and colour profiles, i.e. a gingiva template having gingiva information written on it that was determined using the method according to the invention. Alternatively, the analysis according to FIG. 8 can just as well be detected by means of digital methods, such as photographing or intraoral scanning. The simplest option is the detection of the gingiva status or of the gingiva status and indexing device by means of the photo or film function of a smart phone, iPod or similar device. The data can be stored and transferred in known manner, such as, for example, by ftp, email, storage medium. The digital data of the first digital data set thus obtained can then be processed further as described above.

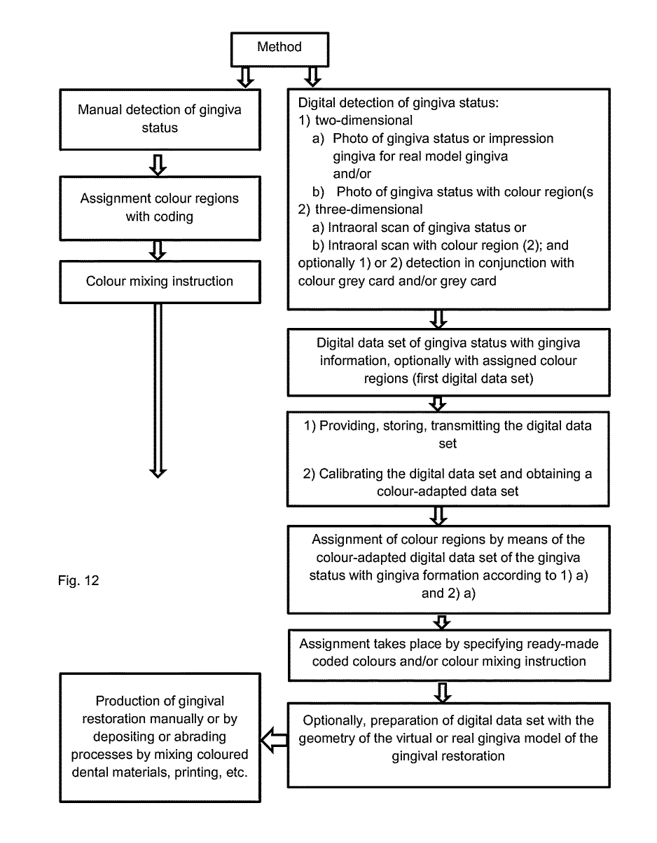

FIGS. 11 and 12 show possible workflows of methods according to the invention for detection of the gingiva status, up to the preparation of the gingival restoration. FIG. 12 discloses the method according to the invention involving the use of a colour grey card or grey card.

### LIST OF REFERENCE NUMBERS

A: width gingiva-dental arch; B: height gingiva-dental arch, C: Contour between the teeth; height and position of the papilla, D: Measure of visibility of tooth stump, E: black triangle, F: Morphology
0 Gingiva indexing device
1 Element, in particular planar element
2 Colour region, in particular coloured body
3 Coding, coded colour region
4 Write-on gingiva template
4.1 Write-on gingiva template of the upper front tooth region,
4.2 Write-on gingiva template of the lower front tooth region,
4.3 Write-on gingiva template of the upper lateral tooth region
4.4 Write-on gingiva template of the lower lateral tooth region
5 Transferred colour region
6 Gingival restoration (FIG. 3)
7 Gingiva status (gingival deficit; gingival recession, missing part of the gingiva) (FIG. 4)
8 Base of the indexing device and/or colour grey card and/or grey card

1. A method for detecting the gingiva status, the method comprising
A) analyzing at least one region of the gingiva status taking place with respect to its shades of red, the position of the shades of red and/or the colour profile of the shades of red and, optionally, the geometry and/or morphology, wherein the analysis is a digital analysis, and
B) gingiva information is obtained,
wherein analysis A) is performed as a digital analysis, and in which
at least one region of the gingiva status and, optionally, one region of the face is detected while using a colour grey card and/or a grey card for calibration, wherein the region of the gingiva is digitally detected, two- or three-dimensionally, in conjunction with the card, wherein digital gingiva information is obtained as a digital data set, or
two- or three-dimensional detecting at least one region of the gingiva status and, optionally, of the tooth status and, optionally, of a region of the face and at least one colour region with a shade of red of an indexing device takes place, whereby the detection takes place in conjunction with the detection of at least one part of a colour grey card and/or grey card for calibration, wherein digital gingiva information is obtained as a digital data set.

2. The method according to claim 1, wherein the gingiva is analysed digitally and/or manually examined with a software, wherein the gingiva status and, optionally, the tooth status is examined with the software for shades and/or colour profiles, with the gingiva being analysed for
(i) colour profiles of the papillae, crestal, in the region of the sulcus, with, additionally or alternatively, (ii) the geometry of the gingiva being analysed, in particular for the position of the papillae, the crestal status, the width of the tooth, the alveolar ridge and/or the tooth status, such as the visibility of the dental necks and/or the
(iii) the morphology of the gingiva being analysed, and wherein
gingiva information being obtained.

3. The method according to claim 2, wherein analysis (i) takes place digitally through the use of the software, and in which
a white balance and/or balance of the colour distortion in correlation to the colour grey card or grey card is performed through the use of a software on the digital data set of the digital gingiva information, and a colour-adapted digital data set is obtained,
the colour-adapted digital data set of the gingiva status and, optionally, of the tooth status is analysed manually with a cursor or automatically for shades and/or colour profiles, whereby gingiva information is obtained, and, optionally,
the gingiva information and/or the gingiva regions each independently have one or more defined, ready-made, dental colours and/or one or more colour mixing instructions assigned to them.

4. The method according to claim 1, wherein the gingiva information is obtained in the form of a gingiva map.

5. The method according to claim 1, wherein the gingiva information
a) is assigned to digitally coded colour regions, or
b) is designed to manually coded colour regions of an indexing device, wherein a colour mixing instruction is assigned, each independently, to the coding.

6. The method according to claim 1, wherein a virtual or real gingival restoration is prepared, wherein
(i) in a step prior to the preparation of the virtual or real gingival model, a first digital data set comprising digital gingiva information of a first virtual model of the gingiva status is prepared by means of intraoral scanning, in conjunction with the detection of a colour grey card and/or grey card for calibration and, optionally, at least one colour region with a shade of red of an indexing device; alternatively, a first digital data set is prepared via a 3D scan of a silicone impression or via a 3D scan of a real model and each is supplemented with gingiva information, and, optionally,
(i.1) performing a white balance and/or a balance of the colour distortion and obtaining a colour-adapted digital data set, and, optionally,
(i.2) analysing the colour-adapted data set for shades and/or colour profiles, and obtaining gingiva information and, optionally,
(i.3) assigning the gingiva information to ready-made dental colours and/or a colour mixing instruction, and
(ii) preparing the digital data set with the geometry of the virtual or real gingiva model of the gingival restoration and gingiva information, and, optionally,
(iii) producing a gingival restoration.

7. A method for producing of a virtual or real model of a gingival restoration comprising the method according to claim 1, and comprising step C) producing a virtual or real model of a gingival restoration.

8. A gingiva indexing device, wherein
the device comprises a) at least one colour region, whereby the colour region comprises at least one shade of red, and b) the indexing device has at least one colour grey card and/or one grey card assigned to it.

9. The device according to claim 8, wherein the device comprises
b) at least one region, in which a colour grey card and/or grey card is arranged.

10. The device according to claim 8, wherein a colour mixing instruction is assigned to the colour regions.

11. A kit comprising the gingiva indexing device according to claim 8 and a write-on gingiva template.

12. A gingival restoration obtainable according to the method according to claim 1.

13. A digital data set obtainable according to the method according to claim 1 a data carrier.

14. Software configured for processing digital data sets obtained according to the method according to claim 1 and/or for preparing digital data sets of the gingival restoration.

15. The software according to claim 14, wherein the software allows for analysis of digitally supplied data of the at least one region of gingiva for its shades of red, the position of the shades of red, the colour profile of the shades of red and/or the morphology, and provides gingiva information, and, optionally, for assigning coded colour regions, wherein a colour mixing instruction is assigned to the coding, and/or for calculation of digital data sets of a gingival restoration.
